(19) United States
(12) Patent Application Publication
(10) Pub. No.: US 2022/0096857 A1
(43) Pub. Date: Mar. 31, 2022

(54) Device for Non-Invasive Treatment of Diseases and Conditions of Living Organisms

(71) Applicant: Synergotron, D.O.O., Zagreb (HR)

(72) Inventors: Domagoj Prebeg, Zagreb (HR); Milan Kordic, Zagreb (HR); Domagoj Liebl, Zagreb (HR); Zvonimir Rudomino, Sesvete (HR)

(21) Appl. No.: 17/421,009

(22) PCT Filed: Dec. 31, 2019
(86) PCT No.: PCT/HR2019/000031

§ 371 Date: Jul. 6, 2021

(30) Foreign Application Priority Data
Jan. 11, 2019 (HR) P20190078A

(51) Int. Cl. A61N 2/00; A61N 1/44; A61N 5/06; A61N 2/02

(52) U.S. Cl. CPC A61N 2/002; A61N 1/44; A61N 5/0613; A61N 2/02; A61N 2005/0626; A61N 2005/0643; A61N 2005/0667; A61N 2005/0651; A61N 2005/0653; A61N 2005/0661; A61N 2005/0662; A61N 2005/0659; A61N 2005/0644

(57) ABSTRACT

The inventors have developed a device for non-invasive treatment of diseases and conditions of living organisms (10) and a methodology for the use of plasma electrophysical stimulation coupled to resonance for the synchronous and synergistic application of several different physical stimuli, including light, electromagnetic field, electric current, dielectric barrier discharge, micro-vibrations and sound, which can operate at different cellular or tissue levels for the purpose of extended and more comprehensive stimulation of the target treated tissue. These factors are generated locally at the site of application, and the electromagnetic field along with electric currents allow a non-invasive application of stimulation by establishing a therapeutic resonant energy pathway (108) between the points of application of the device on the surface of the treated organism by affecting deeper located parts of the organism. In doing so, the resonance effects of these factors can be achieved by adjusting impulse profiles used for their generation. The present device also enables real-time monitoring of the treated tissue response, as well as dose control, and precise positioning of the impulse profile sources during treatment.

### FIELD OF THE INVENTION

The present invention relates to the field of devices for non-invasive treatment of diseases and conditions of living organisms and the methodology of application of such devices for non-invasive treatment of diseases and conditions of living organisms, more specifically, to the device and the method for the synchronous and synergistic use of frequency- and amplitude-modulated pulse profiles, light, electromagnetic field, electric current, dielectric barrier discharge and its constituents, micro-vibrations, sound and with the generation of a therapeutic resonant energy pathway through the target tissue for the purpose of treating diseases and conditions of living organisms.

### Technical Problem

Modern science confirms that every cell in the human body, of more than 100 trillion cells, generates, absorbs, communicates and responds to various forms of electromagnetic energy and vibration, and that nothing happens in a living organism without electromagnetic or vibrational exchange. There is a constant interaction between our cellular electromagnetism and vibrations, and those imposed from the environment. The principle of resonance implies cellular absorption of the introduced electromagnetic energy and their vibrations or their harmonics. In this way, interfering frequencies can be imprinted into cells, which can be the consequence of toxic substances, traumas, pathogens or prolonged exposure to contamination. Disruption of electromagnetic energy and vibrations can cause damage to cell metabolism and low cellular energy, regardless of the initial cause. The principle of therapeutic application of resonance implies cellular absorption of the introduced electromagnetic energy and vibrations or their harmonics for harmonization, i.e. revitalization of cell metabolism and restoration of cellular energy, physiological function and establishing homeostasis.

Electromagnetism, high-frequency currents and dielectric barrier discharge have been used for more than a century as effective therapeutic factors and energy carriers, which, among many properties, are capable of eliminating pathogens and stimulating innate regenerative processes of living organisms.

The biological effects of electromagnetic fields are attributed to thermal and non-thermal mechanisms. Most thermal effects (heating) include increased metabolic activity and vasodilation with increased perfusion of the treated tissue. The most common non-thermal mechanism for electromagnetic fields and the tissues is the effect of cellular excitation of ion exchange (movement of calcium and other ions) across voltage channels that are sensitive to oscillating electric fields. Disturbances in the function of neuronal calcium signaling are known to be associated with inflammatory and degenerative processes of nerve tissue. Calcium metabolism has also been recognized as an important factor involved in cell regenerative processes. The common use of electromagnetic field stimulation is associated with the treatment of edema, erythema, inflammatory processes, arthralgia, bone and wound healing, pain reduction and an increase in joint mobility.

The therapeutic use of currents on tissues has been shown to activate cellular organelles responsible for cellular activity and cellular energy levels, i.e. to increase the concentration of ATP in cells. Ion channels in the cell membranes can migrate under the influence of electric currents, which leads to cytoskeletal modifications including the formation of membrane projections that allow cell movement. This can facilitate the cell proliferation and protein synthesis, which has been demonstrated in cases where electro-stimulation is being applied to skin, tendons, and cartilage and bone cells. The common use of electrical stimulation is associated with treatment in physical therapy for relaxation, stimulation and rehabilitation of muscles, prevention of atrophy, increase of local blood circulation (arterial, venous and lymphatic flow), maintenance and increase of movement, reduction of chronic, persistent, post-traumatic and postoperative acute pain, immediate post-surgical muscle stimulation to prevent venous thrombosis and promote wound healing.

There are biological molecules that absorb the light of wavelengths in the ultraviolet, visible and infrared portion of the spectrum. These molecules, known as chromophores, act together as little antennas that can receive an electromagnetic wave of a passing photon. If the resonant structure of the molecule corresponds to the photon wavelength, the absorption of the photon is possible. Molecules such as hemoglobin, vitamin B12, cytochrome C and many others are examples of chromophores in human cells. When they absorb the light of a certain wavelength, such molecules activate or produce the energy necessary for their function or activity. These processes are recognized as photoactivation or photobiomodulation. The light absorption in the infrared portion of the spectrum can lead to a local increase in temperature; activations of flavin and cytochrome in the mitochondrial respiratory chain with a consequent effect on electron transport; production of reactive oxygen species through activation of endogenous porphyrins and photoactivation of calcium channels with increasing intracellular calcium concentration and subsequent cell proliferation. The use of ultraviolet light spectrum shows bactericidal properties, improved nucleic acid synthesis and increased oxygen consumption in mitochondria. The common use of photostimulation is associated with the treatment of skin disorders, psoriasis, acne vulgaris, eczema, chronic wounds, and reduction of painful symptoms and stimulation of wound healing.

A dielectric barrier discharge is used in the production of cold atmospheric plasma. Cold atmospheric plasma is an ionized gas that is generated by electromagnetic energy. It consists of resonant electrons, photons and other energized particles that create a unique electromagnetic and micro-vibrational trace. Cold atmospheric plasma has been used for therapeutic purposes for more than a century, and millions of treatments have been performed by such a method because of its antimicrobial and bio-stimulating properties. Studies in the field of plasma medicine indicate obvious interactions between treated tissues and constituents of cold atmospheric plasma. These interactions include antimicrobial effects, control of cellular properties and matrix of tissue, activation of fluids and formation of surface and intracellular electric fields. There is ample evidence that controlled application of cold atmospheric plasma can deactivate dangerous pathogens, including those that are resistant to multiple types of antibiotics, stop bleeding or improve wound healing processes without damaging healthy tissue. The common use of cold plasma is associated with decontamination of skin and wounds from pathogenic microorganisms, promoting wound healing, skin regeneration, and revitalization, treatment of skin diseases and arrest of bleeding.

There is evidence that all living cells generate their micro-vibrations and can absorb externally imposed vibrations, more precisely mechanical or acoustic impulses. Therapeutic use of sound and micro-vibrations is associated with treatment in physical therapy to increase blood supply, lymphatic drainage, and tissue oxygenation, improve cellular metabolism, remove cellular and waste tissue products, reduce swelling and scarring, relieve tissue tension and alleviate painful symptoms.

Inventors believe that resonance, and synchronous and synergistic stimulation with multiple physical factors (entrainment), can be considered as the most important principle explaining the effects of devices and methods of application on living organisms. Resonance can be described as the frequency of vibration that is natural to a particular organ or body. There is a constant interaction between the natural frequencies of the cells and those imposed by the environment. The principle of resonance implies cellular absorption of the applied factors of light, electromagnetic field, electric current, and micro-vibration or their harmonics. Resonance is engaged to coordinate cells that are imprinted with interfering/destructive frequencies that may result from toxic substances, trauma, pathogens or prolonged exposure to contamination.

As each living organism, organ, tissue, cell, molecule or microorganism has a unique electromagnetic imprint, inventors believe that they can be treated with a specific resonant frequency to influence their behaviour. Through the simultaneous and synergistic application of different physical stimuli, more specifically, light, electromagnetic field, electric current, dielectric barrier discharge, micro-vibrations, and sound, with the principle of resonance entrainment can be achieved, i.e. harmonization of disturbed electromagnetism and vibration of cells, in order to increase their energy balance and revitalization at different cellular and tissue levels, or pathogenic microorganisms can be eliminated from the body.

For this purpose, the inventors have developed a device and methodology for the application of plasma electro-physical stimulation coupled with resonance for the synchronous and synergistic application of several different physical stimuli, including light, electromagnetic field, electric current, dielectric barrier discharge, micro-vibrations and sound, which can act on different cellular or tissue levels for the purpose of a controlled, expanded and more comprehensive stimulation of the target treated tissue. These factors are generated locally at the site of application, and the electromagnetic field and electric currents allow the non-invasive application of stimulation by establishing a therapeutic resonant energy pathway between the points of application of the device on the surface of the treated organism by acting on deeper located parts of the organism. In doing so, the resonant effects of these factors can be achieved by adjusting the impulse profiles used to generate them. The device also enables real-time monitoring of treated tissue response, dose control, and precise positioning of impulse profile sources during treatment.

### PRIOR ART

Tesla describes in the patent GB189620981A resonance transformer circuits that have made it possible to use naturally oscillating forms of high-frequency electromagnetic energy in medicine. Tesla's circuit is based on coupled primary and secondary transformers, capacitors and a spark. The circuit operates in a cycle with current from the primary transformer connected to the capacitor, which is then discharged through the spark gap. This creates a transient, oscillating current in the primary circuit that causes the high oscillating voltage across the secondary coil. Cold atmospheric plasma can be generated by the high voltage generated across the ends of the secondary coil. The pulse oscillations in Tesla's device last for milliseconds. Each spark across the spark gap produces an impulse of a damped high-voltage sinusoidal waveform over the secondary coil contacts.

Strong in the patent US733343A was first to describe a device containing a vacuum tube having a breakdown electrode in combination with a high-voltage power source.

Floyd describes in the U.S. Pat. No. 2,326,773 an applicator that generally contains evacuated tubes with atmospheric air in which electrical discharge can be produced, and the atmosphere inside the tubes is such that it produces ultraviolet radiation (U.S. Pat. No. 2,326,773).

The same principle is described by Mylius in patent WO2006119997A2, WO2006072582A1 in the construction of an ozone-producing therapeutic device that uses partially evacuated tubes with the addition of noble gases.

Korous and Srb describe in the patent WO2014032747A1 a device for treating biological tissue with low-pressure plasma, where the device comprises a transformer for generating a high-frequency electromagnetic field, a probe that can be electrically coupled to a transformer, and a control unit for controlling the high-frequency electromagnetic field generated by the transformer. In accordance with the invention, the probe is connected to an information unit, wherein the unit is used to measure, and optically and/or acoustically displays the duration, output power of the device and/or measures output current power of the device, and if a pre-defined limit value is exceeded, interrupts power supply to the device; and/or measures and displays the optical and/or acoustic distance of the probe treatment surface from the target tissue surface. In the shown setup, it is obvious that no system defines or measures the distance of electrode from the treated tissue surface, and that the system does not include feedback from the treated area, but only the feedback on the current power, i.e. power on the transformer primary.

The prior art also indicates the necessity of electrically driven vibrators and massagers that produce a vibration that stimulates circulation in the affected tissue. Also known in the art are vibration and impact devices that promote bone growth.

For example, in the U.S. Pat. No. 5,273,028 McLeod describes a device for stimulating bone growth in a living organism by transmitting vertical vibrations through the plate on which the person is located.

In the U.S. Pat. Nos. 5,103,806, 5,376,065 and 5,191,880 McLeod also describes a method for preventing osteopenia while promoting bone growth and healing, including broken bones by subjecting the bones to mechanical loading.

In the U.S. Pat. No. 6,245,006 Olson describes magnetic therapy as an established and reliable technology. In the U.S. Pat. No. 5,632,720, Kleitz describes a magnetic massage with a motor that, when used, does not come closer than 18 inches to the human body. Therefore, the device does not need to come into physical contact with the body. The wand uses a magnetic field between 950 and 1050 gauss in intensity to facilitate an increase in blood flow.

The U.S. Pat. No. 6,602,275 reveals to Sullivan the use of scattered photon light waves at 470 nm, 630 nm, and 880 nm to stimulate the process of human healing by reducing the inflammation, stimulating and rebalancing the electromagnetic field surrounding the lives, detox organs, and tissue.

The U.S. Pat. No. 5,035,235 by Chesky discovers the use of musical sound waves as a therapy for chronic and acute pain.

The U.S. Pat. No. 5,645,578 by Daffer et al. also describes a therapeutic device that uses musical tones.

In the U.S. Pat. No. 7,335,170B2 Milne and Spawr describe a therapeutic micro-vibration massage device, which generates a dynamic magnetic field force and an electromagnetic photon optical light field, followed by acoustic sound penetrating the human body, stimulating an increase in cellular energy, and thereby stimulating a healing effect that reduces or eliminates pain. The device consists of an engine that produces micro-vibrations and sound signals and drives one or more permanent magnets or electromagnets and one or more light sources. The device does not generate high-voltage impulses and high-frequency currents in its operation and is intended solely for local application to the target treated tissue.

Some of the previous devices use one or two thematic technologies. The main disadvantage of all these therapeutic devices is that they are limited to local/topical stimuli application without the ability to control and adjust impulse profiles during the therapeutic procedure, and which, the inventors believe, plays an important role in the outcome of treatment. More specifically, all of the described devices produce and control only some of the above factors, using fixed impulse profiles, which limits applicability, that is, makes it impossible to apply the resonance principle during treatment. The aforementioned devices also do not have a clearly defined control of energy flow, i.e. real-time dosing and monitoring of treated tissue response during stimulation used during treatment, and which depend on a number of factors including the method of administration and deposition of therapeutic electrodes on the surface of the target treated tissue, conductivity and permeability of the treated tissue as well as other ambient conditions such as humidity and air temperature, which play a significant role in the therapeutic outcome of the treatment and/or condition repeatability of the therapeutic procedure. With this in mind, it is understandable that none of the conventional technologies provides a non-invasive technique for the controlled simultaneous stimulation of a living organism by light, electromagnetic field, electric current, dielectric barrier discharge, micro-vibrations a sound-specific impulse profiles with the possibility of applying the resonance principle for non-invasive treatment of a living organism. Accordingly, in the technical field remains a need for techniques to control energy flow-dosage, modulation of the electromagnetic impulse profile, and monitoring of tissue response to stimulation used during real-time treatment, as well as controlled indirect stimulation by creating a resonant circuit through specific structures, pathways and/or body areas for the purpose of treating various conditions and diseases without introducing a physical invasion of a living organism.

None of the aforementioned devices utilizes or suggests simultaneous, synergistic application of different waveforms of frequency- and amplitude-modulated high-voltage impulse profiles, electromagnetic field, electric current, dielectric discharge, light and micro-vibrations, and do not allow the application of a resonant circuit through the target treated tissue. The prior art is therefore characterized by numerous drawbacks, which are explained in the solutions of the present invention. The solutions of this invention minimize, and, in some cases, eliminate the aforementioned disadvantages, and integrate the technology into a practical hand-held or desktop device. Besides, the solutions disclosed in the present invention offer a more cost-effective production of certain elements, more specifically, transducers, increase the safety and repeatability of the therapeutic procedure and reduce energy consumption both in production and during application of the device itself, compared to the devices shown so far. Conducted lab and clinical studies indicate that the dose, i.e. amounts of energy transmitted, as well as the waveform itself, frequency and amplitude of the output high-voltage signal, current intensity and voltage, transducer position in relation to the target treated surface, as well as the physical properties of the target treated tissue itself, or structures to which the device disclosed by the present invention is applied, play a significant role in the outcome of therapeutic interventions.

### BRIEF SUMMARY OF THE PRESENT INVENTION

The device disclosed by the present invention provides a method for applying plasma electro-physical stimulation and the establishment of a therapeutic resonant energy pathway through the target treated tissue for treating diseases and conditions of living organisms. This approach is non-invasive and does not involve physical interference with the treated organism. The therapeutic effect of the device and the method of application includes increasing the energy balance of the target treated tissue and matching electromagnetism and vibration of cells to their natural frequencies through synchronous synergistic application of physical stimuli to adaptive impulse profiles including light in the infrared, visible and/or UV portion of the spectrum, electromagnetic field, electric current, dielectric barrier discharge, micro-vibrations and sound, and the formation of a therapeutic resonant energy circuit of specific impulse profiles (current, electromagnetic) through specific regions, pathways or tissue structures.

The device for non-invasive treatment of diseases and conditions of living organisms disclosed herein enables the application of frequency- and amplitude-modulated impulse profiles via a pair of therapeutic electrodes that allow simultaneous application of light, EM fields, electric current, micro-vibrations, sound and dielectric barrier discharge by establishing a therapeutic resonant energy pathway through a target treated tissue containing at least one resonant electrode and at least one transducer, wherein the resonant electrode enables the establishment of a therapeutic resonant energy pathway, and the measurement of transmitted energy, voltage, and impedance current during the treatment to control the dose and to adjust the output impulse profile to the generator and impulse profiles controller according to the type and response of the target treated tissue.

The device comprises one or more pairs of therapeutic electrodes, namely transducers and resonant electrodes. One or more transducers are placed directly on the skin or close to the skin of the organism at specific locations, and serve as a source that allows selected stimuli and impulse profiles designed to treat certain diseases or conditions to be applied to a living organism. One or more resonant electrodes are placed directly on the skin or in the immediate vicinity of the skin of the organism at specific locations in such a way that the electromagnetic energy is guided through the treated organism, more precisely by forming a therapeutic resonant energy pathway between one or more transducers and one or more resonant electrodes. In this way, the formation of a therapeutic resonant energy pathway is controlled by the placement of a transducer and a resonant electrode on a living organism, and provides guided stimulation through a specific region (e.g. joint, muscle, limb or abdomen) or via certain conductive pathways in a living organism (e.g. the innervation area of a particular nerve, energy meridians or reflex points). In this way, specific stimuli can be introduced both locally and systematically, i.e. through certain regions of a living organism.

In addition, the resonant electrode serves as a measuring probe that allows real-time monitoring of delivered energy during treatment, i.e. dosing and monitoring of tissue response (voltage, current, impedance) to stimulation used during treatment.

The device acts on the living organism in five basic ways. It should be noted, first of all, that through the target treated tissue located between the transducer and the resonant electrode the electric current (at biological proportions) of a relatively high frequency of high-voltage impulses flows, which allows the formation of a therapeutic resonant energy pathway, whose properties can be adjusted according to the purpose of the therapeutic procedure. In this way, the adaptation of the impulse profiles can result in resonance with different types of tissues for their targeted excitation, focusing, or dispersion of electromagnetic field and applied energy or for the elimination of microorganisms by the principle of mortal oscillating resonance. The second effect creates an electromagnetic field generated by the transducer. The third effect comes from the absorption of light transmitted by the transducer. The fourth effect is created by the dielectric barrier discharge and its constituents, which are created in the space between the active surface of the transducer and the treated surface. All these factors cause micro-vibrations, as a fifth effect, which affect the target treated tissue, while generating dielectric barrier discharge in the field of work, additional sound waves are generated whose properties depend on the characteristics of the high-voltage signal impulse profile used.

The device produces impulse profiles and stimulation factors that are defined by a therapeutic regimen configured to achieve a therapeutic effect for the treatment or suppression of a specific disease or condition of a target treated tissue. Application of the device includes therapeutic treatments: injuries of soft and hard tissue; pain; reflex, trigger and acupressure and acupuncture points and pathways; neurological and neuromuscular disorders and injuries, peripheral neuropathy and circulatory disorder, acute and chronic injuries and disorders of the locomotor system, hyperplasia and neoplasia, inflammations and infections, or elimination of microorganisms.

The therapeutic effects of the present device include revitalization, stimulation of regenerative processes and pain reduction including enhancement of blood and lymph circulation, tissue oxygenation, repolarization of cell membranes, activation of differentiated and undifferentiated cells, reduction of swelling and edema; facilitation of cellular metabolism, energy production, enhanced ion exchange and elimination of metabolites and waste from cells and tissues; reduction of the number of microorganisms, stimulation of the immune response, arrest of bleeding, or pain reduction.

The various embodiments of the devices described in the present invention also allow for their minimisation, simplification and cost reduction in manufacturing, control and targeted application of specific light spectra and electromagnetic imprint transmitted to the target treated tissue, and allow control of transducer positioning relative to the surface of the target treated tissue in case of dielectric barrier discharge application, which allows the control of the properties and the increased efficiency of the constituents of the dielectric barrier discharge generated therein, more specifically, enables the adjustment of the distance between the transducer and the surface of the treated organism and the volume at which the discharge is generated. This also reduces the diffusion of cold atmospheric plasma constituents from the site of application to the environment and consequently reduced effective concentration at the site of application, which has a significant effect on the end therapeutic result, especially in the case of microorganism elimination, arrest of bleeding or topical stimulation of the surface layers of target treated tissue by dielectric barrier discharge.

For example, the invention may also be applied to the topical application of physical stimuli of different impulse profiles including light in the infrared, visible and/or UV portion of the spectrum, electromagnetic field, electric current, micro-vibrations, sound and dielectric barrier discharge and its constituents on the skin surface and mucous membranes, or open wounds or exposed body parts, such as subcutaneous tissue, muscle, bone, etc., to eliminate microorganism, stimulate healing and reduce pain.

Furthermore, the invention can be applied to stimulate one or more areas of target tissue of a living organism by placing one or more transducers directly on the skin surface in the projection or in the immediate vicinity of the target treated tissue, and placing one or more resonant electrodes on the skin at the desired point in order to form a therapeutic resonant energy pathway. In that case, the position, i.e. spatial arrangement of the transducers and resonant electrodes in relation to the treated organism, the triggering method (parallel or serial) in the case of applying multiple transducers and resonant electrodes, and the properties of the used impulse profile depend on the topology and purpose of the therapeutic intervention itself. Coupling one or more transducers to one or more resonant electrodes allows the formation of a therapeutic resonant energy circuit through the body between the points of their application. Such an application allows the electromagnetic impulse profiles and stimuli to be directed through specific tissue structures or pathways (nerves, energy meridians, or reflex zones) or areas of the body (joints, muscles, limbs, head, neck, chest or abdomen).

The number of application points of transducers and resonant electrodes can vary from one point to twenty points on a living organism, and the treatments can be applied once or twice a day for an extended period of weeks, months or years. The duration of such a procedure at the point of application can vary from a few seconds (10-60 sec) in the case of applying dielectric barrier discharge up to 30 minutes in the case of applying a resonant circuit through a particular region of a living organism (e.g. knee).

The device for non-invasive treatment of diseases and conditions of living organisms disclosed herein comprises a central unit for the production and control of high-voltage impulse profiles, and one or more pairs of therapeutic electrodes, where each pair of therapeutic electrodes contains one transducer and one resonant electrode, and wherein a central unit is coupled to the referred above one or more pairs of therapeutic electrodes, where the device for non-invasive treatment of diseases and conditions of living organisms generates the course of high-voltage impulse profiles, which are transmitted to the living organism via a pair of therapeutic electrodes, where the transducer acts as a source and the resonant electrode as a sink in the specified system. The central unit may be in the form of a portable hand-held or desktop unit, and it comprises enclosures, a battery or mains power source, a management unit, a control unit, a voltage stabilizer, a block of a generator and a signal profile regulator, which is a block of high-voltage signal profiles. The signal profile generator and controller unit comprises a high-voltage transformer, an energy block connected to the transformer primary, and a measuring block connected to an energy block, a resonant electrode and a management unit, with at least one transducer connected to the secondary of the high-voltage transformer.

The management unit receives a (measurable) voltage signal from the block of the generator and signal profiles regulator, which is generated on the basis of the voltage and current measured within the transducer and the resonant electrode, and on the basis of these measurements, a voltage control signal (modulation) is generated whose amplitude is proportional to the current energy transmitted to the therapeutic resonant energy pathway, and based on monitoring this signal, the control unit can determine in real time whether the disease and condition treatment device is working properly and whether the predicted amount of energy for the particular treatment has been delivered. Waveform voltage parameters on the secondary of the high-voltage transformer of the block of the generator and signal profile regulator are variable and adjustable so that the impulse waveform, amplitude, frequency, amplitude- and frequency modulation in time can be changed and adjusted including the frequency of impulse sequences (impulse frequency), frequency of modulated impulses (resonant frequency) and frequency of modulated signal (amplitude-modulated frequency) and depth of amplitude modulation.

The impulse waveforms that make up the impulse sequence can be square, sinusoidal, trapezoidal, triangular, sawtooth, reversed sawtooth, linear increase (rumpup), linear decrease (rumpdown), exponential growth (rumpup), exponential decay (rumpdown), even harmonics, odd harmonics, exponentially damped sine, exponentially amplified or damped sine, or modulated impulse width.

Besides, the voltage on the secondary of the high-voltage transformer of the block of the generator and the signal profile regulator is modulated by one of the above waveforms, whereby the depth and the frequency of the amplitude modulation are adjustable.

For example, it is possible that the voltage on the secondary of the high-voltage transformer is generated as a sine wave (FIG. 7a-c) or as a square (FIG. 7d-f) waveform of 43.2 kHz modulated by a sine wave (FIGS. 7a and d), a square (FIGS. 7b and e) or a sawtooth (FIGS. 7c and f) waveform of a control signal voltage (modulation signal) of 2.16 kHz, with a modulation amplitude depth of 80%.

These are just a few examples of the combination of the signal waveform and modulation signal. In doing so, the device can use any combination of the above waveforms for both impulse generation and generation of the voltage control signal (modulation signal).

Different therapeutic impulse profiles consisting of impulse sequences (impulse arrays) that can be regulated and modulated with respect to the waveform (sinusoidal, rectangular, trapezoidal, linear or exponential growth or decay, even and odd harmonics, damped sine), impulse sequence frequency from 0.1 Hz to 7,500 Hz with the frequency modulation in specified range, resonant frequency from 20 kHz to 350 kHz, and voltage amplitude from 500 V to 30,000 V with voltage amplitude modulation by oscillating signal in the range from 0.1 Hz to 5,040 Hz with an adjustable modulation depth can be applied via management unit.

When the transducer is in proximity to a living organism and when the device for non-invasive treatment of diseases and conditions of living organisms is in operation, it enables the formation of a dielectric barrier discharge, generating an electromagnetic field in the frequency range from 40 MHz to 1 GHz and micro-vibration, i.e. a sound in the frequency range from 0.1 Hz to 20 kHz, and a dielectric barrier burst and a mildly ionized cold atmospheric plasma are generated in the air gap between the transducer and the treated tissue surface. When the device for non-invasive treatment of diseases and conditions of living organisms is used to treat the tissue, a transducer—treated tissue—resonant electrode coupling is established, whereby the block of generator and signal profile regulator in real time measures the amount of energy, voltage, current and impedance during treatment implementation, that serves to monitor the amount of energy delivered and the response of the treated organism to stimulation, which depend on the administration modality, the type of target treated tissue and/or ambient conditions during administration allowing real-time control of the amount of energy delivered, by adjusting the output impulse profiles, created by the block of generator and signal profile regulator.

A transducer typically comprises a connector, an insulating housing, a capacitive and/or inductive element, a dielectric barrier, a light source that generates a smoldering discharge in a partially evacuated volume enclosed by dielectric containing one or more gases, which are excited over a dielectric barrier through connectors by means of a capacitive element coupled to the generator and the signal profile regulator. In another embodiment, the transducer comprises a connector, an insulating housing, capacitive and/or inductive element, a dielectric barrier, an electronic circuit with a LED or OLED light source, or an alternative light source in the UV, visible or infrared portion of the spectrum, excited by separate impulse profiles generated by the management unit. The dielectric barrier in the transducer is usually made of glass, ceramics or polymers. The capacitive and/or inductive element is made of conductive material either as a capacitive plate or as a network, or a coil, or a combination thereof, which also may be transparent to light, and is located on the proximal side of the dielectric barrier.

$$\frac{1 + \sqrt{5}}{2}$$

The inductive elements contain a contact point for source and sink coupling, the inductive element being directly coupled to the block of generator and impulse profile regulator, and wherein the operation of the device does not require a direct contact with the treated organism, but the transducer and the device are placed at a certain distance from the organism for generating a therapeutic field.

The active surface of the transducer is in the form of a dielectric barrier that is directed or comes into contact with the treated organism, made flat, convex, concave, pointed, or combination thereof, in order to satisfy energy and ergonomic requirements, all depending on the topology and the type and needs of the therapeutic procedure, and is made of glass, ceramic or polymer.

The transducer may further comprise adaptive extensions for precise positioning of the transducer relative to the treated surface of the living organism, it may further comprise a telescopic extension having an inner wall, and may also include a dielectric photo filter, whereby during the operation of the device it is possible to control the discharge properties and to retain constituents generated by the occurrence of dielectric barrier discharge within a closed volume whose walls comprise the active surface of the transducer, the treated surface of the living organism and the inner wall of the telescopic extension, wherein the dielectric photo filter covers the active surface of the transducer transmitting a specific spectrum of light onto the target treated tissue. The adaptive extension may also comprise passive elements adjacent to the active surface of the transducer (506) and the target treated surface, which allow the same distance to be maintained for use on larger surfaces of the living organism.

The resonant electrode comes in contact with the surface of the organism, wherein the said electrode is made of conductive material of arbitrary dimensions and shapes, and is coupled to a central unit, wherein the resonant electrode comprises an electronic circuit that enables voltage measurements and visual or audible signaling to establish a therapeutic energy resonant circuit.

The device for treatment of diseases and conditions disclosed herein generates impulse profiles that include impulse sequences that can be adapted and modulated with respect to their shape, which may be either a sinusoidal, a rectangle, a triangle, a sawtooth or other suitable shape, with impulse frequency ranging from 0.1 Hz to 7,500 Hz, with a resonant frequency ranging from 20 to 350 Hz, with the impulse width ranging from several hundred nanoseconds to 20 microseconds, wherein the amplitude modulation by an oscillating signal is in the frequency range from 0.1 to 5,040 Hz, wherein the impulse amplitude is in the range from around 500 V to 30 kV, and wherein electromagnetic field and high-frequency currents ranging from 40 MHz to 1 GHz and sound waves in the range from 0.1 to 20,000 Hz are generated through the generation of a dielectric barrier in the air gap between the transducer surface and the surface of the target treated tissue. In specific embodiments one or more specific frequencies or sweep over the specified frequency range are used. The depth of the amplitude modulation ranges from 1 to 100%.

Electromagnetic transduction and mechanical transduction are such processes in which cells in a living organism convert electromagnetic and mechanical stimuli into biochemical signals. Tissue and cells can absorb and respond to different types of electromagnetic and mechanical stimuli. The effects of stimulation depend on the type and property of the stimuli. The inventors have found that the simultaneous, harmonised and controlled application of multiple physical stimuli to different pulse profiles including light in the infrared, visible and/or UV portion of the spectrum, electromagnetic field, electric current, dielectric barrier discharge and its constituents, micro-vibrations and sound provides a comprehensive and synergistic stimulation of biochemical signals as well as enhanced effect of cellular entrainment at different cellular and tissue levels, which significantly improves the outcome of the therapeutic intervention. The present invention also enables the application of therapeutic procedures based on the principle of establishing a therapeutic resonant energy circuit through the target treated tissue that allows non-invasive application of various impulse profiles on deep tissue structures placed below, i.e. in the projection of the treated surface of a living organism or through certain conductive pathways below the surface of a living organism including nerves, blood vessels, energy meridians and points.

### BRIEF DESCRIPTION OF THE DRAWINGS

FIG. 1a. Two basic derivatives of the device for the application of the plasma electrophysical stimulation and resonance

FIG. 1b. A schematic of the therapeutic device

FIG. 2. Description of basic control signals

FIG. 3. Mode of signals u_{K1}, u_{mod} and Uₙₐₚ

FIG. 4. Waveform voltage U_{VN} on a transducer amplitude-modulated by the sinusoidal waveform u_{mod}

FIG. 5. U_{VN} voltage on the transducer-increased time axis

FIG. 6. Representation of waveform voltage U_{VN} and waveforms of the modulation signal u_{mod}: 1. square, 2. sinusoidal, 3. trapezoidal, 4. triangular, 5. sawtooth, 6. reversed sawtooth, 7. linear increase (rumpup), 8. linear decrease (rumpdown), 9. exponential growth (rumpup), 10. exponential decay (rumpdown), 11. even harmonics, 12. odd harmonics, 13. exponentially damped sine, 14. exponentially amplified sine and 17. modulation of impulse width

FIG. 7. Embodiment examples of modulating the sinusoidal and square waveform of a signal by the sinusoidal, square and sawtooth waveforms of the modulation signal u_{mod} (at base signal frequency=43.2 kHz, modulation signal frequency=2.16 kHz and amplitude modulation depth (AM) 80%)

FIG. 8. Measuring the amount of the transferred energy

FIG. 9. Electrical equivalent scheme of the system shown in FIG. 8

FIG. 10. A view of some typical transducer embodiments

FIG. 11. A view of some potential inductive transducer elements

FIG. 12. Contact forms of the transducer active surface and the treated surface

FIG. 13. Adaptive transducer extension

FIG. 14. Administration modalities of the device

FIG. 15. A representation of couplings of transducers and resonant electrodes and the establishment of the therapeutic resonant energy pathway through the target treated tissue of a living organism

### A LIST OF REFERENCE SIGNS USED IN DRAWINGS

8—hand-held device
10—device for non-invasive treatment of diseases and conditions of living organisms
112—multichannel source of electromagnetic impulse profiles
100—central unit
101—power supply source
102—management unit
103—control unit
104—power supply stabilizer
105—block of the generator and signal profile regulator
106—transducer
107—resonant electrode
108—therapeutic resonant energy pathway
109—dielectric barrier discharge
110—target treated tissue
204—output impedance R_{gVN}
206—inductance L_{gVN}
208—capacity C_{gVN}
210—transducer impedance Zₜᵣₐₙₛ
212—current that flows through the target tissue impedance Iₜⱼ
214—target tissue impedance Zₜⱼ
216—resonant electrode complex impedance Z_{rez}
218—generator current output I_{VN}
220—generator voltage output U_{VN}
222—signal processing in the measuring block of the generator and signal
profile regulator
401—insulating housing
402—capacitive or inductive element
403—smoldering gas discharge
404—dielectric barrier
405—connector for coupling to the generator and impulse profiles regulator (source)
406—retention element
407—electronic circuit with a light source
408—light source (in the UV, visible or infrared portion of the spectrum)
409—connector for coupling of the light source to the management unit
410—connector for coupling of the inductive element sink to the generator and impulse profile regulator
501—adaptive transducer extension
505—retention element on the transducer housing
506—active surface of the transducer
507—telescopic extension
508—dielectric photo filter
509—passive element for maintaining a constant distance when used on larger surfaces
601—single-contact capacitive plate for source coupling
602—single-contact capacitive disc for source coupling
603—single-contact capacitive network for source coupling
604—dual inductive disc with a dual coupling (source-sink)
605—inductive coil with a dual coupling (source-sink)
701, 607—contact point for source coupling (block of the generator and impulse profile regulator)
702, 608—contact point for sink coupling (block of the generator and impulse profile regulator)
700—therapeutic field
801—flat shape of the transducer tip
802—convex shape of the transducer tip
803—concave shape of the transducer tip
804—pointed shape of the transducer tip

### DETAILED DESCRIPTION OF AT LEAST ONE WAY OF CARRYING OUT THE INVENTION

There are two basic embodiments of this device for non-invasive treatment of diseases and conditions of living organisms (10) through the application of plasma electrophysical stimulation and resonance, as a hand-held device (8), which in principle consists of one transducer (106) and one or more resonant electrodes (107), and as a desktop device that consists of a multi-channel source of high-voltage impulse profiles (112) coupled to one or more transducers (106) and resonant electrodes (107). It is a device for synchronous and synergistic use of frequency- and amplitude-modulated impulse profiles, light, electromagnetic field, electric current, micro-vibrations, sound, and dielectric discharge barrier and its constituents with the generation of a therapeutic resonant energy pathway through the target tissue to treat diseases and conditions of living organisms. The present device is non-invasive, and more specifically, does not involve physical penetration of parts of the device into an organism, but is applied solely on its surface or in its immediate vicinity. The device generally consists of a central unit (100) which can be configured as a portable hand-held (8) or desktop device for the production and control of high-voltage impulse profiles, and is coupled to one or more pairs of therapeutic electrodes, more specifically, to a source, i.e. transducer (106) and a sink, i.e. resonant electrode (107). The purpose of the device is to generate a sequence of high-voltage impulse profiles that are transmitted to a living organism via a pair of therapeutic electrodes, with the transducer acting as a source and resonant electrode playing the role of a sink in the aforementioned system.

In one of its embodiments the present device (10) is applied on specific areas of the body using certain impulse profiles that generally include impulse sequences, which can be adjusted and modulated with respect to their shape (sine, right triangle, sawtooth, etc.), frequency of impulse sequences (burst) in the frequency range from 0.1 Hz to 7,500 Hz, through one or more specific frequencies or sweep over the specified frequency range, resonant frequency (of modulated impulses) in the frequency range from 20 to 350 Hz, through one or more specific frequencies or sweep over the specified frequency range, and amplitude modulation (of modulation signal) by an oscillating signal in the frequency range from 0.1 to 5,040 Hz, through one or more specific frequencies or sweep over the specified frequency range, impulse amplitude in the range from about 500 V to 30 kV, and with amplitude modulation depth in the range from 1% to 100%. The treatment also involves the application of an electromagnetic field, high-frequency currents in the range from 40 MHz to 1 GHz and sound waves (0.1 to 20.000 Hz) through the generation of a dielectric barrier discharge in the air gap between the surface of the transducer and the surface of the target treated tissue.

The central unit (100) comprises housings, a battery or mains power source (101), a management unit (102), a control unit (103), a power supply stabilizer (104), and the block of the generator and signal profile regulator (105), which is in fact a source of high-voltage signal profiles.

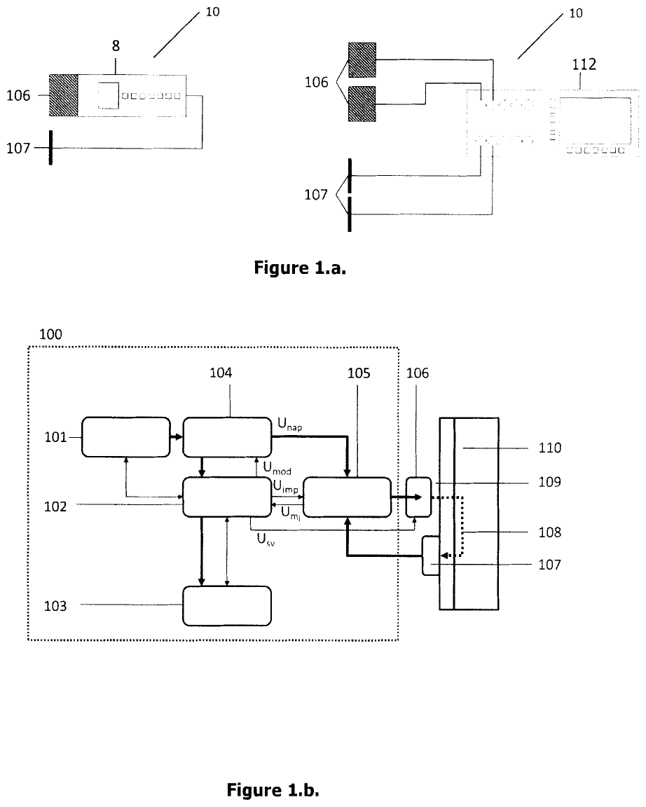

For the proper and efficient operation of the device, it is necessary to ensure the control of the energy amount that is transferred to the treated living organism during treatment. To this end, guidance signals are generated, marked by the arrows in FIG. 1 as well as one additional within the controller. In this case, control of the energy flow will mean control over the shape, frequency, amplitude, and duration of the high-voltage impulse profiles, i.e. impulse sequences.

The power supply source, i.e. energy source (101) in FIG. 1 my be a galvanically isolated power supply connected to the electrical network or a battery with associated charger and charge control circuits. The power supply stabilizer (104) comprises multiple circuits for generating voltages of different values necessary for supplying other circuits of the system with power. These voltages are fixed in time, but the one indicated in FIG. 1 as Uₙₐₚ that serves for supplying the block of the generator and impulse profile regulator (105) with power may also be variable in time. The amount of voltage Uₙₐₚ depends on the managing signal u_{mod} generated by the control unit (102). Variation in the amplitude of this signal will allow variations in power supply voltage Uₙₐₚ, which allows for variations in the amplitude of high-voltage impulses. The amplitude of high-voltage impulses is also affected by the Uᵢₘₚ signal, which controls the energy block at the source of high-voltage impulses. One or more power blocks are connected to one or more high-voltage transformers on whose secondary a transducer is connected. Managing signals are also generated from the management unit block towards the control unit (103), and the management unit (102) also monitors feedback signals from the control unit (103). In this way, it is possible to select stimulation parameters (amplitude, frequency, and amplitude and frequency modulation of signal profiles), to start and stop the stimulation, and to monitor the course of stimulation on the intended display units. The management unit (102) also communicates bidirectionally with the power supply source (101). This achieves battery control, or in the case of mains power supply, control of on-off control systems. The management unit (102) also generates signal U_{SV} that in particular embodiments of the transducer (106) controls the intensity of light therein.

A particularly important signal is the Uₘⱼ received by the management unit (102) from the measuring block (222) of the generator and signal profile regulator (105). It is created based on the voltages and currents measured inside the transducer (106) and the resonant electrode (107). Based on these measurements, the Uₘⱼ signal whose amplitude is proportional to the instantaneous energy that is transmitted to the therapeutic resonant energy pathway (108) is created. Based on the monitoring of this signal, the control unit can determine in real time whether the system is operating as intended and whether the intended dose for the selected treatment has been delivered. Based on this signal, it is possible to change in real time the other aforementioned signals. This achieves the regulation of the energy amount transmitted in a unit of time to the therapeutic resonant energy pathway.

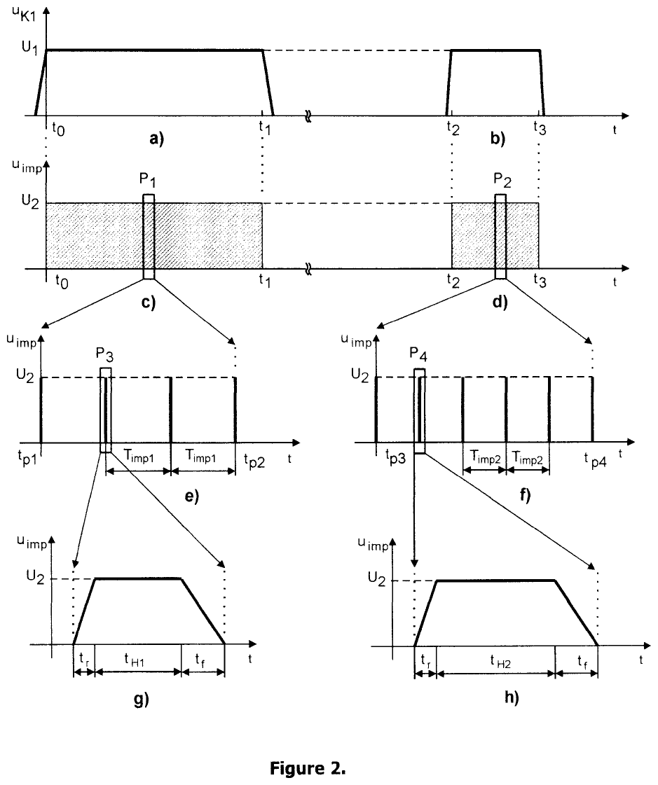

FIG. 2 shows basic control signals within the control unit (102). u_{K1} signal at the top of FIG. 2 (Figures a and b) is the main guiding signal. It is a voltage signal whose activation can be seen as a voltage increase from zero to a value U₁. Activation is initiated by the user himself by starting the system operation on the control unit (103). This is how the sequence of treatment begins that in FIG. 2a lasts from moment t₀ to the moment t₁. The interval between these two moments in absolute amount is determined by the user before activation, by selecting the settings according to the purpose and requirements of the treatment, and is defined by the total amount of the transmitted energy and can vary in the interval from ten seconds to ten minutes. Once the treatment sequence is complete, the device waits for reactivation. It occurs in FIG. 2b at the t₂ moment and the treatment lasts up to the t₃ moment. The treatment duration is not the same as the one from the previous activation, because it was assumed that the user changed the settings of the treatment in the interval between the moments t₁ and t₂. The interval between moments t₁ and t₂ is arbitrary and user-dependent. The user may arbitrarily discontinue the treatment before its expiration on the control unit (103). The layout of the u_{K1} signal will be the same as in FIGS. 2a and 2b, only the interval between the voltage rise and fall will be smaller.

The high u_{K1} signal level will initiate the generation of the Uᵢₘₚ signal. It is the signal, which manages the block of the generator and signal profile regulator (105) on the transducer shown in FIGS. 2c and 2d in the time scale from FIGS. 2a and 2b. To be able to see the characteristics of this signal, it is necessary to increase the time scale. This is done by defining the time windows P₁ and P₂ and their representation in new FIGS. 2e and 2f with an increased time scale. The arrows connecting the Figures indicate the same points in time.

Uᵢₘₚ signal is actually a sequence of impulses, as seen in FIGS. 2e and 2f. The impulses are spaced by a time interval Tᵢₘₚ₁, i.e. Tᵢₘₚ, considering that Tᵢₘₚ₁>Tᵢₘₚ so the frequency of occurrence is lower in the former stimulation sequence. The time interval between the impulses is determined by the user via the control unit (103) and is related to the length of treatment. The longer the treatment, the bigger the allowed Tᵢₘₚ value that can be set, i.e., the frequency of the impulse appearance will be smaller, as shown in FIGS. 2e and 2f. The values of time intervals Tᵢₘₚ, which can be set, range from a few hundreds of nanoseconds to a few tens of milliseconds. During one treatment the Tᵢₘₚ may have a fixed and/or variable value, i.e., it enables frequency modulation.
To be able to more accurately observe the characteristics of uᵢₘₚ signal, it is necessary to increase the time scale. This is done by defining the time windows P₃ and P₄ and their representation in new FIGS. 2g and 2h with an increased time scale. The arrows connecting the Figures indicate the same points in time.
On an enlarged scale, it can be observed that the signal has three separate parts. In the first part, the voltage rises from zero volts to U₂ during rise time tᵣ. It then keeps the voltage value at U₂ in t_{H1} duration and then falls to zero volts at the fall time t_{f}. It is visible in FIGS. 2g and 2h that the rise times tᵣ and fall times t_{f} are always the same no matter the values of other characteristic times. What the user can change, is the duration of the signal plateau t_{H}.
The duration of the plateau is not related to other times and it is possible to select all the offered t_{H} values in any combination of other parameters. This fact is emphasized in FIGS. 2e and 2g, as well as in 2f and 2h where it is seen that the shorter Tᵢₘₚ interval does not condition that t_{H} must be shorter. The opposite case has been shown.
In one embodiment of the device is the uᵢₘₚ signal, the signal that is transmitted between the gate and the source of the energy MOSFETs in the high-voltage impulse generation circuit. The characteristics of MOSFETs together with the high-voltage transformer and transducer (106) at the output determine the required rise and fall times that are typically in the range of values from a few tens of nanoseconds to a couple of hundred nanoseconds. Since these times are conditioned by the design of the device, there is no reason for them to be under the user's control.

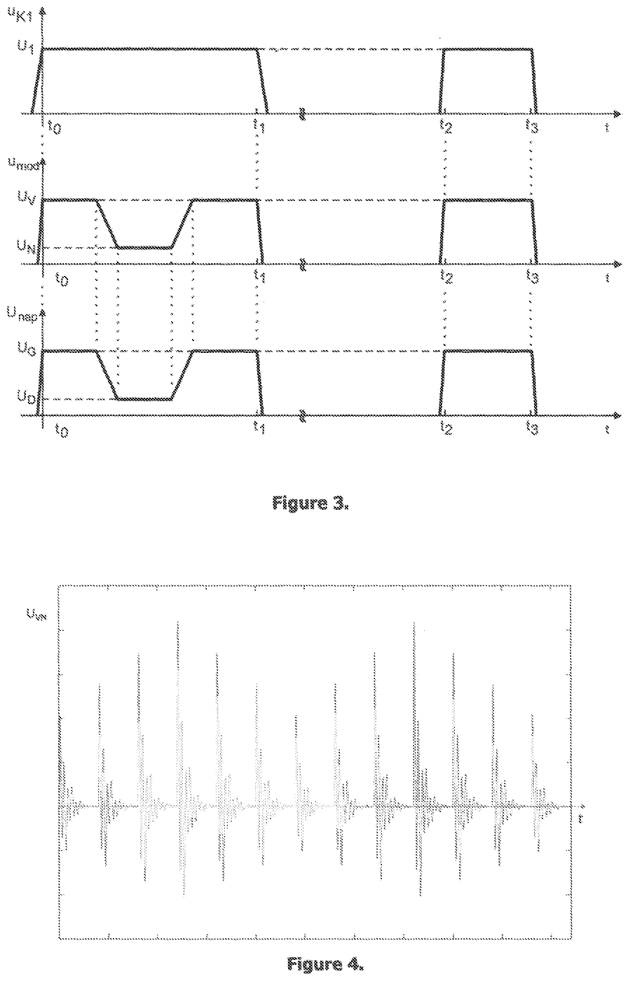

It has already been explained how the signals u_{mod} and Uₙₐₚ are related, and FIG. 3 shows in detail, what their temporal relations are to each other as well as to the u_{K1} signal. The Figure shows that the signals u_{mod} and Uₙₐₚ are in time, with the Uₙₐₚ amplitude being directly related to the u_{mod} amplitude with some coefficient, which means that the Uₙₐₚ voltage is amplitude modulated. In FIG. 3 the u_{mod} signal has a broken waveform in the interval from t₀ to t₁, which maps to the same one at Uₙₐₚ, meaning that an arbitrary form of voltage can be generated (e.g. rectangle, exponential growth and decay, linear increase and decrease, sine, etc.). Examples of waveforms are shown in FIG. 6. The limitation lies in the fact that the voltage changes of u_{mod} are not too fast concerning the final rate of speed change, which may be followed by the voltage stabilizer control loop provided by Uₙₐₚ. For practical implementation, this means that changes can take place for several hundred nanoseconds or more.

FIG. 3 also shows that the U_{mod} signal simultaneously serves as an enable signal of the block of the generator and signal profiles regulator (105). The design of the power supply stabilizer (104) enables this, which gives an important safety component to the operation of the device. It should be noted that the Uₙₐₚ signal is an energy signal, which means that the energy portion of the device will remain out of power supply when the U_{K1} signal is inactive. Therefore, even if there is a uᵢₘₚ signal when the u_{K1} signal is inactive due to a fault, the high-voltage impulse source remains without the possibility of energy transfer to the user.

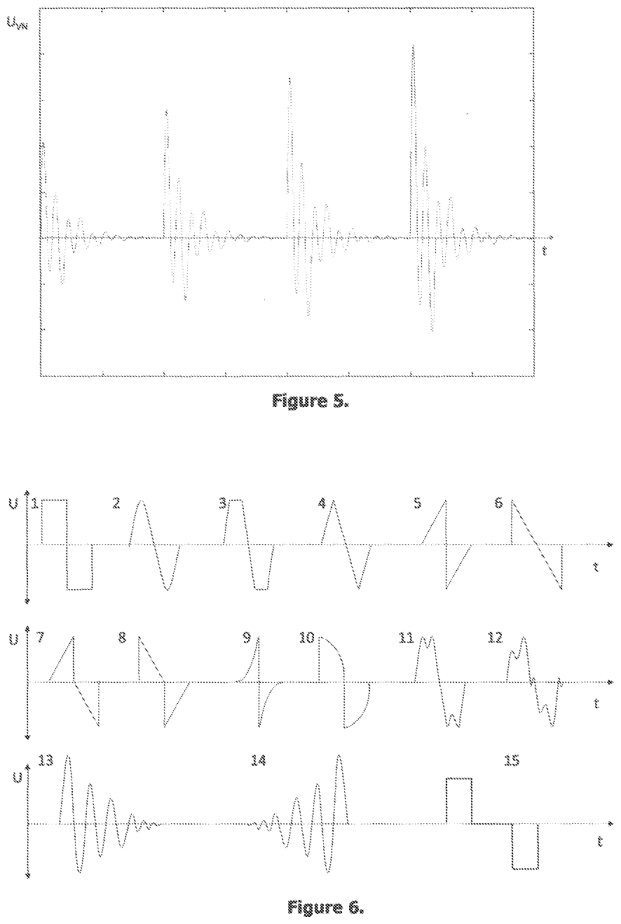

The layout of the energy signal U_{VN} on the transducer (106) in one embodiment of the device, can be seen in FIG. 4. This Figure shows the result of the device operating when the u_{K1} signal is active. The Figure shows the use of a sinusoidal waveform of the modulation signal u_{mod}, in the embodiment of a device with a naturally oscillating resonance transformer circuit, more specifically, with an open-core transformer. A more detailed insight into the possible voltage and impulse form in such a device embodiment can be seen in FIG. 5. The impulse form can be described by an exponentially damped sinusoidal voltage. The reason for this form lies in the fact that the secondary of the high-voltage transformer together with the secondary parasitic capacity of the transformer freely flickers. The energy is first stored in the transformer, while the MOSFET is switched on in the primary circuit, i.e. by t_{H} (FIGS. 2g and 2h), and then the transistor shuts off and energy is transferred to the secondary circuit. Since there are losses in the secondary winding in this oscillator, damping occurs in the electrode and in the resistance of the body of the user, which is seen in the exponential decline of oscillation amplitudes with time. It should be emphasized that most of the damping comes from the resistance of the secondary winding, so the voltage waveform will depend a little on the electrical characteristics of the treated living organism. Therefore, the amount of the energy transmitted to the user body will vary slightly (on the order of 10%) on a case-by-case basis. FIG. 5 shows an edge case where the distance between individual Tᵢₘₚ impulses (FIGS. 2e and 2f) is set to start the next impulse at the moment when the energy of the last impulse has already been transferred and consumed.

The moment of MOSFET activation on the primary can easily be observed by the positive voltage spike at the beginning of the waveform shown in FIG. 5. The uᵢₘₚ voltage duration is (tᵣ+t_{f}+t_{H}) and can vary from a few hundred nanoseconds up to 20 μs. Depending on which transistor is switched on on the primary, the initial spike may be either positive or negative, which also changes the polarity of the voltage of the first period of oscillation, and thus the polarity of the modulated signal envelope (FIG. 4), which allows the system operation to be adjusted according to the goals and requirements of the therapeutic intervention.

In another embodiment of the device, the implemented block of the generator and signal profiles regulator (105) has full control over secondary voltage. It also has guidance signals, as described so far, with the difference that the voltage waveform parameters on the transformer secondary (amplitude, frequency, and modulation of frequency and amplitude in time) are variable and adjustable. This means that, at its output, it is possible to obtain both the waveforms of FIGS. 4 and 5, but also the waveforms shown in FIG. 6, except that the waveforms do not depend on the parasitic elements of the secondary circuit of the transformer. The waveforms shown in FIG. 6 also represent the waveforms of the u_{mod} modulation signal. FIG. 6 shows the waveforms of U_{VN} voltage and the waveforms of the modulation signal u_{mod}: 1. square, 2. sinusoidal, 3. trapezoidal, 4. triangular, 5. sawtooth, 6. reversed sawtooth, 7. linear increase (rumpup), 16. linear decrease (rumpdown), 9. exponential growth (rumpup), 17. exponential decay (rumpdown), 11. even harmonics, 12. odd harmonics, 13. exponentially damped sine, 14. exponentially amplified sine and 15. modulation of the signal width.

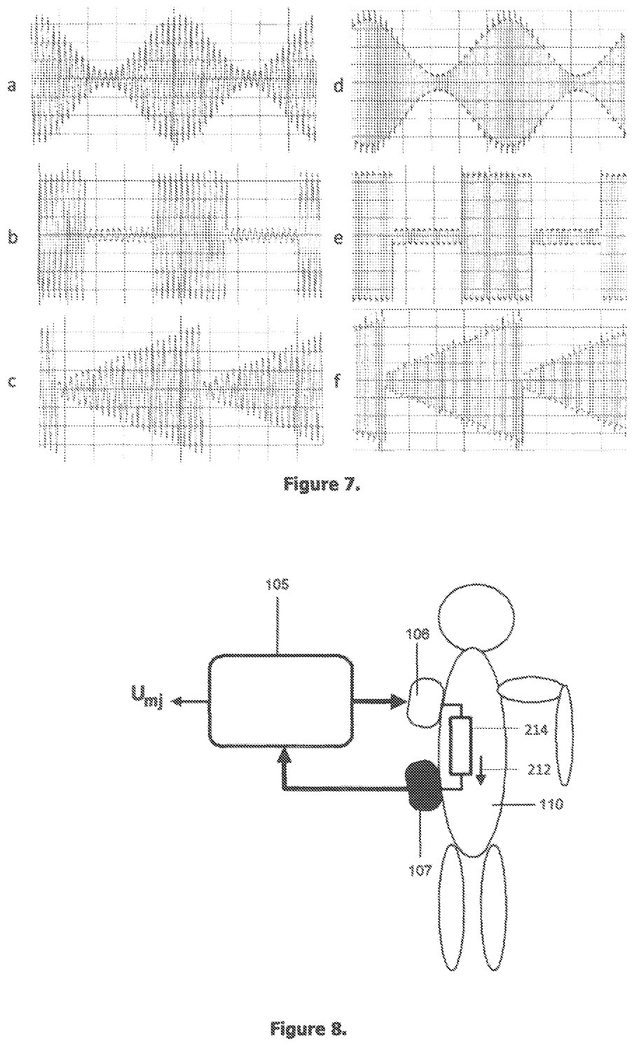

FIG. 7 shows some of the modulation designs of the sine wave signal Uᵥₙ by sinusoidal (7a), square (7b) and sawtooth (7c) waveforms of the modulation signal u_{mod}, as well as the modulation designs of the square wave Uᵥₙ by sinusoidal (7d), square (7e) and sawtooth (7f) waveforms of the modulation signal u_{mod}. With the ability to select the characteristics of Uᵥₙ and u_{mod}, the described system can adjust the amplitude depth ranging from a just few percents to 100%. In particular, FIG. 7 shows an example of the modulation design of a sinusoidal and square waveform of a signal by the sinusoidal, square and sawtooth waveforms of the modulation signal u_{mod} (at base signal frequency=43.2 Hz, modulation signal frequency=2.16 Hz, and amplitude modulation depth (AM) 80%).

The amount of transmitted energy will depend on the duration of the described basic signals. How the energy is transmitted, i.e., how much energy is transmitted in a unit of time will depend on the amplitude and frequency modulation signal and its characteristics. The user does not have complete control over all combinations, but only over those allowed by the controller to avoid possible overuse of energy in the unit of time.

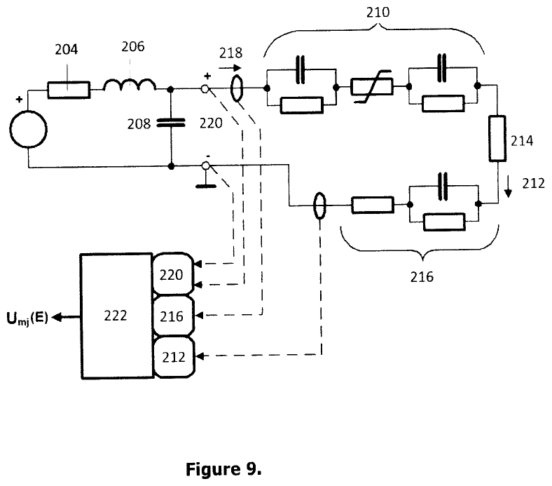

Measuring the amount of the transferred energy is reduced to measuring the target tissue impedance Zₜⱼ (214) and current Iₜⱼ (212) that flows through this impedance as shown in FIG. 8.

The block of the generator and signal profile regulator (105) provides energy that passes through the transducer (106), the target treated tissue (110) and the resonant electrode (107).

An equivalent electrical scheme of this part of the system is shown in FIG. 9. The output circuit of the generator and signal profile regulator is replaced by the ideal voltage source U_{gVN} and by the output passive network of elements. Output impedance R_{gVN} (204) represents the secondary impedance of the high-voltage transformer as well as the mapped impedance of the primary circuit. Inductance L_{gVN} (206) and capacity C_{gVN} (208) in the same way represent the parasitic inductance and secondary capacity of the high-voltage transformer, as well as the mapped inductance and capacity of the primary circuit. It can be said with great certainty that these elements will be linear and independent of the currents and voltages in the network. This does not apply to the transducer impedance Zₜᵣₐₙₛ (210) that has a dominant element of the voltage-dependent nonlinear impedance. The voltage dependence physically arises from the ionization of the low-pressure gas contained within the electrode. The resonant electrode has the same complex impedance Z_{rez} (216), which, however, is predominantly determined by small series impedance. Therefore, it can be assumed that there is practically a short circuit at the stimulation frequency on the electrode. With this assumption, we can say that the current passing through the resonant electrode will be practically equal to the current passing through the target tissue impedance Iₜⱼ (212).

The value of the voltage signal Uₘⱼ is proportional to the transferred energy. To determine this, it is necessary to measure the U_{VN} voltage and the current at the generator output I_{VN} (218) and signal profile regulator, and the reverse current at the output of the resonant electrode Iₜⱼ (212). These two currents differ in value and phase due to the parasitic capacities that exist between all nodes in the equivalent scheme. Based on the measured amplitudes and voltage phases, as well as the amplitude and phase currents, it is possible to calculate the body impedance Zₜⱼ (214). Based on its value, it is also possible to determine the energy transferred to the treated tissue.

From the above considerations, it can be seen that most of the impact on the target tissue will come from the direct effects generated by the current flow and the dielectric barrier discharge along with the transducer. Other effects expected from the electromagnetic field, sound and light will manifest through a minor change in the impedance of the target tissue. Therefore, measuring voltages U_{VN}, I_{VN}, and Itj as accurate as possible is crucial. An extremely high value of interference will be present when measuring due to the immediate vicinity of the generator and signal profile regulator. Several electrostatic shielding measures have been used to minimize the impact of these interferences. When installing the shielding, it was taken into account not to create new parasitic capacitive pathways that would transmit energy from the generator to the mass and that bypass the target tissue. In addition to shielding, a differential signal measurement is used as another way to suppress interference. Interferences also appear here as a single-phase signal at the input of the measurement channel, which is designed for a specially extended single-phase signal travel at the input. Extended signal travel was not sufficient to protect against high amplitude interference, so special attention was given to the circuit protection of the measuring channel. They must meet the opposing requirements for maintaining the large input impedance of the measuring channel and, on the other hand, small physical dimensions that are consistent with the size of electrodes.

The central unit (100) is coupled to one or more transducers (106), i.e. sources that convert electromagnetic impulse profiles into the light, electromagnetic field, electric current, dielectric barrier discharge, micro-vibrations and sound transmitted to the target treated tissue (110). The central unit (100), more specifically, the block of the generator and signal profile regulator (105) is also coupled to one or more resonant electrodes (107), i.e. sinks that enable the establishment of a therapeutic resonant energy pathway (108) through the target treated tissue (110), i.e. between the points of application of the transducer (106) and the resonant electrode (107). The central unit (100), i.e. the block of the generator and signal profile regulator (105) via transducer (106)—treated tissue (110)—the resonant electrode (107) coupling, in real time, measures the amount of energy, more specifically, the voltage, current, and impedance during treatment, thus monitoring the amount of the transmitted energy and the response of the treated organism to stimulation, which depend on the administration modality, the type of target treated tissue and/or ambient conditions during application. This allows for real-time dose control by adjusting the output impulse profiles generated by the block of the generator and impulse profile regulator (105). Impulse profiles and doses for each therapeutic procedure are defined by settings stored in the memory of the management unit (102).

The central unit in the desktop version of the device comprises one or more blocks of the generator and impulse profiles regulator (105) coupled to the management unit (102) on one side and the transducer (106) and the resonant electrode (107) on the other side. In this case, the management unit enables parallel or serial operation (switching on/off) of individual generators and impulse profile regulators.

The impulse profile settings, more specifically, the impulse sequences and their waveforms and doses are adapted to a particular disease or condition of the treated living organism based on clinical experience, and are stored in the management unit (102), and are defined based on the administration modality, the resonant frequency of the individual tissue or, e.g. the microorganism, and the total amount of the transmitted energy.

The device (10) enables the regular application of various therapeutic impulse profiles, generally consisting of impulse sequences (impulse arrays) that can be regulated and modulated by the management unit (102) with respect to the waveform (sinusoidal, rectangular, trapezoidal, linear or exponential growth or decay, even and odd harmonics, damped sine), impulse sequence frequency from 0.1 Hz to 7,500 Hz, impulse width ranging from several hundred nanoseconds to 20 microseconds with the possibility of frequency modulation in the specified range, a resonant frequency from 20 kHz to 350 kHz, and a voltage amplitude of 500 V to 30,000 V with voltage amplitude modulation by oscillating signal in the range from 0.1 Hz to 540 Hz with adjustable modulation depth.

The device (10) also enables the application of a dielectric barrier discharge when the transducer is in immediate vicinity to a living organism generating an electromagnetic field in the frequency range from 40 MHz to 1 GHz and micro-vibrations, i.e. sound in the frequency range from 0.1 Hz to 20 kHz, also a dielectric barrier discharge together with a slightly ionized cold atmospheric plasma are generated in the air gap between the transducer and the surface of the treated tissue.

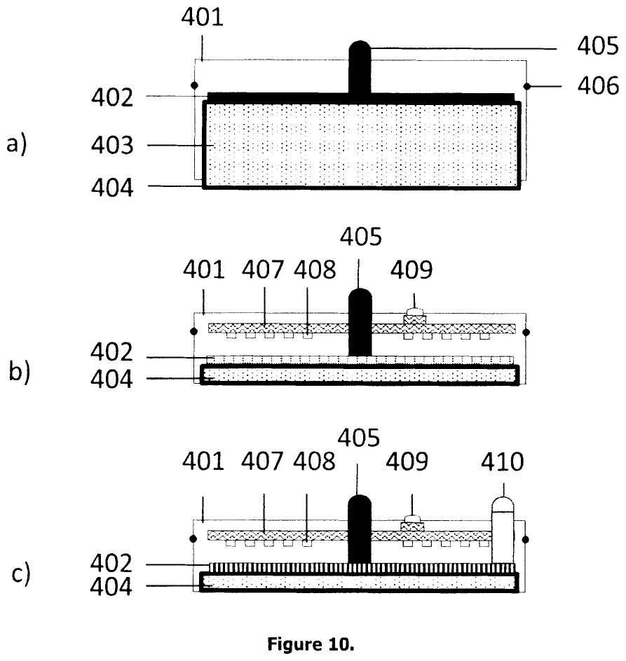

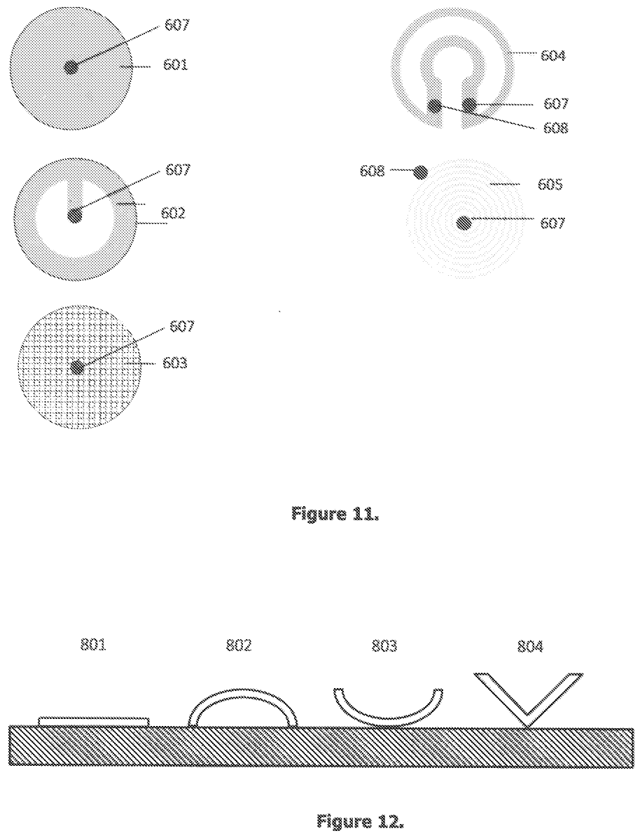

The transducer (106), as shown in FIG. 10, in principle, contains an insulating housing (401), a capacitive and/or inductive element (402), a dielectric barrier (404) made of glass, ceramics or polymers, and a light source generated by a smoldering discharge in a partially evacuated volume enclosed by dielectric containing one or more gases (403), which by means of the capacitive element (402) (601 in FIG. 11) coupled to the block of the generator and signal profile regulator (105) are excited over a dielectric barrier (404) through the connector (405), or the light generates a separate electronic circuit (407) with a light source (408) such as LED, OLED or any other alternative light source in the UV, visible, or infrared portion of the spectrum, excited by separate impulse profiles generated by the management unit (102). Also, FIG. 10 shows a connector for the coupling of the light source to the management unit (409) and the connector for the coupling of the inductive coil sink to the generator (410).

Capacitive and/or inductive element (402), shown in FIG. 10, can be made of conductive material as a capacitive plate (601), a capacitive disc (602) or as a capacitive network (603), an inductive disc (604) or a coil (605) or combination thereof, which may be transparent to light, as shown in FIG. 11, and is located on the proximal side of the dielectric (opposite side of the active surface of the transducer).

A coil with a dual coupling (605), shown in FIG. 11, is made of conductive material and comprises a contact point for source coupling (607) and a contact point for sink coupling (608). A coil (605) can be flat or conically wound and arrangement and length of coil are of arbitrary width/thickness of wire.

In the embodiments of the transducer containing inductive elements (604), (605), as shown in FIG. 11, this element contains a contact point for source coupling (607) and sink coupling (608), and is directly coupled to the block of the generator and signal profile regulator (105), and their operation is possible without a direct contact with the treated organism, whereas the transducer (106) and the device (10) are positioned at a certain distance from the organism to generate a therapeutic field. Some of the possible embodiments of the transducer with inductive elements are a dual inductive disc with a dual coupling (source-sink) (604) or a coil with a dual coupling (source-sink) (605). In the case where the transducer contains capacitive elements, some possible embodiments are a single-contact capacitive plate for source coupling (601), a single-contact capacitive disc for source coupling (602) and a single-contact capacitive network for source coupling (603).

The active surface of the transducer (106), i.e. the dielectric barrier (404), which is directed or comes into contact with the treated organism, can vary in shape and size, all as shown in FIG. 12, wherein the shape is flat (801), convex (802), concave (803) or pointed (804), or combinations thereof, all to meet energy and ergonomic requirements depending on the type and needs of the therapeutic procedure, and is also made of glass, ceramics or polymers.

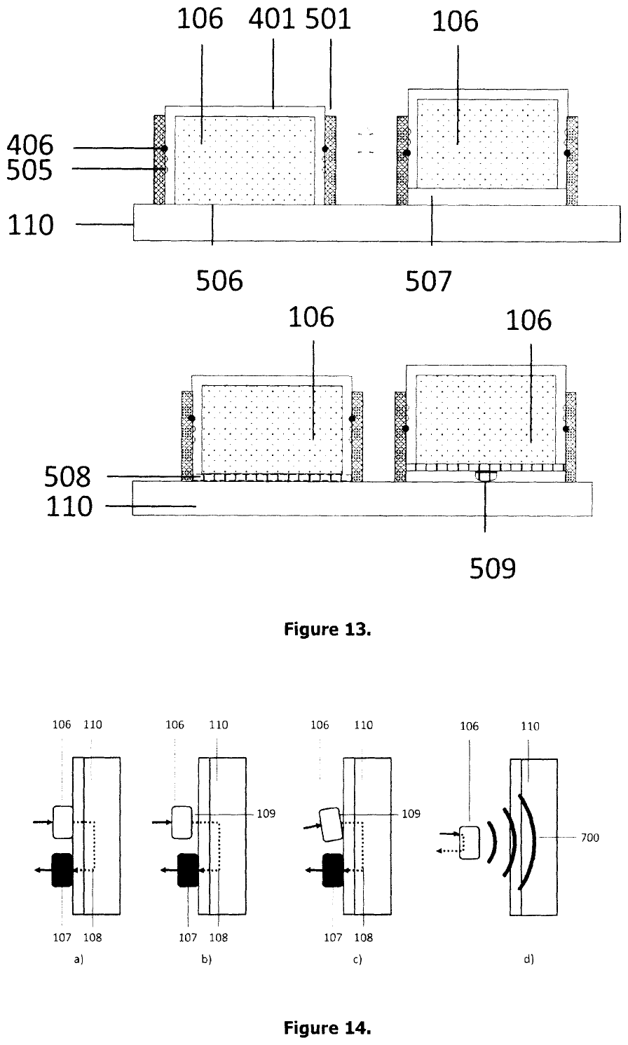

The transducer (106) is usually located in the transducer housing, and may also comprise an adaptive transducer extension (501), as shown in FIG. 13, for precise positioning of the transducer (106) relative to the treated surface of the living organism, more specifically, for defining the distance between the active surface of the transducer (506) and the treated surface of the living organism, it also enables control of the discharge properties and retention of constituents generated by the appearance of a dielectric barrier discharge within a closed volume whose walls comprise the active transducer surface, the treated surface of the living organism, and the inner wall of the telescopic extension (507). The adaptive extension may also comprise a dielectric photo filter (508) that covers the active surface of the transducer for light transmission of a specific spectrum to the target treated tissue, in the case of a wide-spectrum light source usage. The adaptive extension may also comprise passive elements adjacent to the active surface of the transducer (106) and the target treated surface, which allow the same distance to be maintained for use on larger surfaces of the living organism. The transducer (106) may also include a retention element on the extension or a retention element on the transducer housing (505). If necessary, the transducer (106) may also comprise a passive element for maintaining a constant distance when used on larger surfaces (509).

The resonant electrode (107) that comes in contact with the surface of the organism is made of conductive material of arbitrary dimensions and shapes, e.g. a tube or a plate, and is coupled to the central unit (100). The resonant electrode (107) comprises an electrical circuit that allows the U_{M}, measurement described earlier to be performed and visual or audible signaling of the establishment of a therapeutic energy resonant circuit.

In another embodiment, the present device for treatment of diseases and conditions generates impulse profiles that include impulse sequences that can be adjusted and modulated with respect to their shape that can be either square, sinusoidal, trapezoidal, triangular, sawtooth, reversed sawtooth, linear increase (rumpup), linear decrease (rumpdown), exponential growth (rumpup), exponential decay (rumpdown), even harmonics, odd harmonics, exponentially damped sine, exponentially amplified sine or modulated impulse width, with frequencies of the impulse sequences ranging from 0.1 Hz to 7,500 Hz, resonant frequency ranging from 20 to 350 Hz, amplitude modulation by an oscillating signal in the frequency range from 0.1 to 5,040 Hz, impulse amplitude ranging from 500 V to 30 kV with modulation depth ranging from 1 to 100%, wherein electromagnetic field, high-frequency currents ranging from 40 MHz to 1 GHz and sound waves ranging from 0.1 to 20,000 Hz are generated through the generation of dielectric barrier discharge in the air gap between the transducer surface and the target treated tissue surface.

The device is applied in several possible ways, as shown in FIG. 14, by placing one or more transducers (106) and resonant electrodes (107) in direct contact as in FIG. 14a, or immediate vicinity, as shown in FIG. 14b, or combination thereof 14c with the treated surface of the organism at specific sites on the body, and specific impulse profiles designed for treating specific diseases and conditions are applied to or guide through the target treated tissue of the living organism. In the embodiment of the transducer with inductive elements (604 and 605) that are directly coupled to the block of the generator and signal profile regulator (105), a therapeutic field (700) is generated, which affects the treated organism without direct contact between the transducer active surface and the living organism, as shown in FIG. 14d.

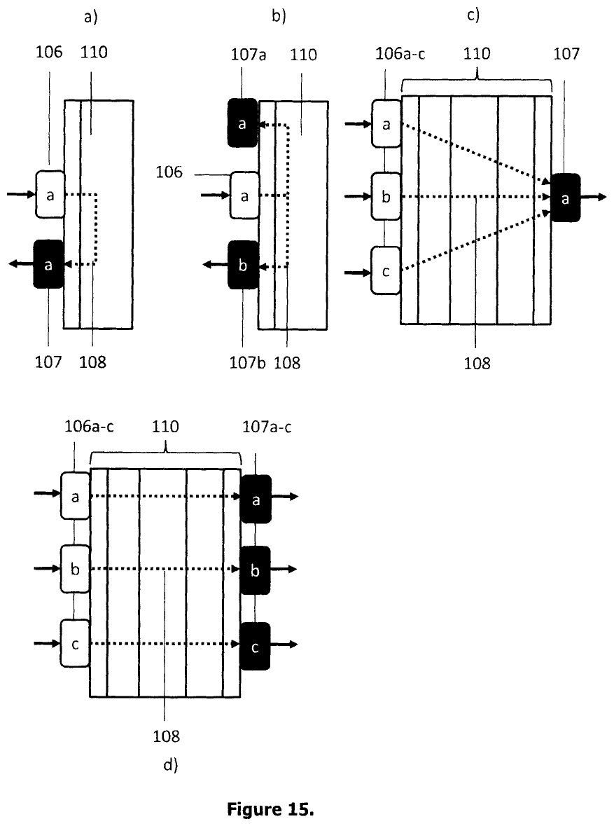

The coupling between one or more transducers (106) and one or more resonant electrodes (107) achieved through the target treated tissue by direct coupling, e.g., when applied to a wound or by capacitive coupling, e.g., when applied to the skin, it enables the establishment of a therapeutic resonant energy circuit, i.e targeted guidance of electromagnetic impulse profiles through target treated tissue, such as joint and muscle, or between target points on the tissue, such as between the reflex points at the upper and lower extremities, to stimulate certain deeper tissue structures, neurological pathways, innervation areas or energy meridians, and centers of a living organism. Some of the possible arrangements of transducers and resonant electrodes in relation to the treated tissue are shown in FIG. 15. In this way, a therapeutic resonant energy pathway circuit may be applied to stimulate a particular pathway on a living organism between two (15a) or more (15b) distant points on the surface, as well as the stimulating deeper structures of a living organism with one or more pairs of transducers and resonant electrodes (15c and d), whereby in the case of multiple pairs of therapeutic electrodes a diffuse or focused effect on a specific target tissue of a living organism may be achieved by controlling the impulse profiles and starting modes (parallel or serial) of individual transducers (FIG. 15d).

1. A device for non-invasive treatment of diseases and conditions of living organisms characterized in that the device comprises a central unit for the production and control of high-voltage impulse profiles, and one or more pairs of therapeutic electrodes, each pair of therapeutic electrodes comprising at least one transducer and at least one resonant electrode, and wherein the central unit is coupled to the one or more pairs of therapeutic electrodes, and wherein the device for non-invasive treatment of diseases and conditions of living organisms generates a sequence of high-voltage impulse profiles transmitted to a living organism by means of the one or more pairs of therapeutic electrodes, the transducer acting as a source and the resonant electrode acting as a sink in the device, whereby the application of frequency- and amplitude modulated impulse profiles is possible via at least one pair of the one or more pairs of therapeutic electrodes allowing simultaneous application of light, EM field, electric current, micro-vibrations, sound, and/or dielectric barrier discharge, along with establishing a therapeutic resonant energy pathway through a target treated tissue, and wherein at least one resonant electrode permits the establishment of the therapeutic resonant energy pathway and measurement of transmitted energy, voltage, and impedance current during treatment to control dose according to a type and a response of the target treated tissue.

2. The device for treatment of diseases and conditions of claim 1, characterized in that the central unit can be designed in the form of a portable hand-held or desktop device.

3. The device for treatment of diseases and conditions of claim 1, characterized in that the central unit comprises one or more housings, a battery or a main power supply source, a management unit, a control unit, a voltage stabilizer, and a block comprising the generator and a signal profile regulator, which represents a source of high-voltage signal profiles, wherein the at least one resonant electrode enables the establishment of the therapeutic resonant energy pathway and the measurement of the transmitted energy, voltage, and/or impedance current during treatment for dose control and adjustment of output impulse profiles on the block comprising the generator and the signal profile regulator according to the type and the response of the target treated tissue.

4. The device for treatment of diseases and conditions of claim 3, characterized in that the block comprising the generator and the signal profile regulator further comprises one or more high-voltage transformers, an energy block attached to the one or more high-voltage transformers, wherein at least one transducer and a measuring block are attached to the energy block, the resonant electrode, and the management unit, and further wherein the at least one transducer and the measuring block are connected to the secondary of the high-voltage transformer.

5. The device for treatment of diseases and conditions of claim 4, characterized in that the management unit receives from the block comprising the generator and the signal profile regulator a voltage signal, wherein the voltage signal is generated based on the voltage and the current measured within the source and the sink, and creating a voltage managing signal based on the current measured within the source and the sink, wherein the amplitude of the voltage managing signal is proportional to the energy, voltage, and/or impedance current transmitted to the therapeutic resonant energy pathway, and wherein the control unit can determine in real time based on signal monitoring whether the device for treatment of diseases and conditions is operating properly and whether an estimated amount of energy for a particular treatment is being delivered or has been delivered.

6. The device for treatment of diseases and conditions of claim 4, characterized in that the voltage waveform parameters on the secondary of the high-voltage transformer of the block comprising the generator and the signal profile regulator are variable and adjustable, so that the amplitude, frequency and amplitude and frequency modulation in time can be changed and regulated.

7. The device for treatment of diseases and conditions of claim 5, characterized in that the waveforms of the modulation signal are square, sinusoidal, trapezoidal, triangular, sawtooth, reversed sawtooth, linear increase, linear decrease, exponential growth, exponential decay, even harmonics, odd harmonics, exponentially damped sine, exponentially amplified sine, modulated impulse width, or a combination thereof, whereby the amplitude modulation depth of the voltage signal is adaptable from 1 to 100%.

8. The device for treatment of diseases and conditions of claim 5, characterized in that the impulse sequences on the secondary of the high-voltage transformer of the block comprising the generator and the signal profile regulator are made of impulses that are square, sinusoidal, trapezoidal, triangular, sawtooth, reversed sawtooth, linear increase (rumpup), linear decrease (rumpdown), exponential growth (rumpup), exponential decay (rumpdown), even harmonics, odd harmonics, exponentially damped or amplified sine, modulated impulse width, or a combination thereof, wherein the frequency impulse modulation is adaptable.

9. The device for treatment of diseases and conditions of claim 5, characterized in that various therapeutic impulse profiles may be applied via a management unit comprising impulse sequences, which can be regulated and modulated according to their waveform impulse sequence frequency, resonant frequency, and voltage amplitude; wherein the impulse sequence frequency is in a range from 0.1 Hz to 7,500 Hz; wherein the resonant frequency is in a range from 20 kHz to 350 Hz and may comprise frequency modulation; and wherein the voltage amplitude is in a range of 500 V to 30,000 V with a voltage amplitude modulation by an oscillating signal in the range from 0.1 Hz to 5,040 Hz with adaptable modulation depth.

10. The device for treatment of diseases and conditions of claim 5, characterized in that when the transducer is in the immediate vicinity of the living organism and when the device is in function, it enables the formation of the dielectric barrier discharge, generating an electromagnetic field in the frequency range from 40 MHz to 1 GHz, and micro-vibrations, wherein micro-vibrations comprise sound in the frequency range from 0.1 Hz to 20 kHz, and generating in an air gap between the transducer and the surface of the treated tissue a dielectric barrier discharge and a slightly ionized cold atmospheric plasma, wherein the central unit is coupled to the transducer.

11. The device for treatment of diseases and conditions of claim 3, characterized in that when the device is used for the tissue treatment, a transducer-treated tissue-resonant electrode coupling is established, whereby a therapeutic resonant energy pathway is established through the target treated tissue, whereby the block comprising the generator and the signal profile regulator measures energy amount, voltage, current, and impedance in real time through the transducer-treated tissue-resonant electrode coupling during treatment to monitor transmitted energy and the living organism's response to stimulation, which depend on the administration modality, the type and condition of the target treated tissue and/or ambient conditions during application, to allow the control of the transmitted energy amount in real time by adjusting the output impulse profiles generated by the block of the generator and signal profile regulator.

12. The device for treatment of diseases and conditions of claim 1, characterized in that the transducer comprises a connector, an insulating housing, a capacitive and/or inductive element, a dielectric barrier, a light source that generates a smoldering discharge in a partially evacuated volume enclosed by dielectric comprising one or more gases that with the help of the capacitive element coupled to the generator and signal profile regulator are excited over dielectric barrier through the connector (405).

13. The device for treatment of diseases and conditions of claim 1, characterized in that the transducer comprises a connector, an insulating housing, a capacitive and/or inductive element, a dielectric barrier, an electronic circuit with a light source of LED or OLED type of light or other alternative light source in the UV, visible, or infrared portion of the spectrum, excited by separate impulse profiles generated by the management unit.

14. The device for treatment of diseases and conditions of claim 12, characterized in that the dielectric barrier in the transducer is made of glass, ceramics, or polymers.

15. The device for treatment of diseases and conditions of claim 12, characterized in that the capacitive and/or inductive element is made of conductive material either as a capacitive plate, a disc, or network, or as an inductive disc, or a coil, or as a combination thereof, and is located on the proximal side of the dielectric barrier.

16. The device for treatment of diseases and conditions of claim 12, characterized in that the capacitive and/or inductive element is made of conductive material such as coil, wherein the coil comprises contact points for source coupling and sink coupling, wherein the coil can be flat or conically wound, and wherein the arrangement and length of flat-coil or conically wound coil are of arbitrary width/thickness of wire.

17. The device for treatment of diseases and conditions of claim 15, characterized in that the inductive element comprises a contact point for source coupling and sink coupling, the inductive element being directly coupled to the block comprising the generator and the signal profile regulator, wherein operation of the device is possible without a direct contact with the treated organism, but the transducer and the device are positioned at a certain distance from the organism to generate a therapeutic field.

18. The device for treatment of diseases and conditions of claim 14, characterized in that the active surface of the transducer is in the form of a dielectric barrier, wherein the dielectric barrier is directed to or comes into contact with the treated organism, is flat, convex, concave, pointed, or a combination thereof to meet energy and ergonomic requirements depending on the type and needs of the therapeutic procedure, and is made of glass, ceramics, or polymers.

19. The device for treatment of diseases and conditions of claim 12, characterized in that the transducer comprises adaptive extensions for precise positioning of the transducer relative to a treated surface of the living organism, furthermore wherein the transducer comprises a telescopic extension with an inner wall capable of precisely defining the distance between the active surface of the transducer wherein the transducer comprises a dielectric barrier and the treated surface of the living organism, thereby allowing, during operation of the device, control of dielectric barrier discharge properties and retention of constituents generated by the appearance of a dielectric barrier discharge within a closed and defined volume whose walls comprise an active transducer surface, the treated surface of the living organism, and the inner wall of the telescopic extension.

20. The device for treatment of diseases and conditions of claim 19, characterized in that the transducer comprises adaptive extensions comprising a dielectric photo filter that covers the active surface of the transducer by transmitting a specific spectrum of light onto the target treated tissue.

21. The device for treatment of diseases and conditions of claim 19, characterized in that the transducer comprises adaptive extensions comprising one or more passive elements for maintaining a constant distance between the dielectric barrier discharge and the surface of the target treated tissue.

22. The device for treatment of diseases and conditions of claim 1, characterized in that the resonant electrode comes in contact with the surface of the organism, and wherein the resonant electrode is made of conductive material and is coupled to the central unit, wherein the resonant electrode comprises an electronic circuit that enables voltage measurements and visual or audible signaling to establish a therapeutic resonant energy circuit.

23. The device for treatment of diseases and conditions of claim 1, characterized in that the device generates impulse profiles that comprise impulse sequences that can be adapted and modulated with respect to their shape, wherein the shape is either sinusoidal, rectangular, triangular, sawtooth, trapezoidal, linear or exponential growth or decay, even or odd harmonics, or exponentially damped or amplified sine with frequency of impulse sequences ranging from 0.1 Hz to 7,500 Hz, wherein the frequency of modulated impulses is in a range from 20 Hz to 350 Hz, wherein the modulation frequency of the modulation signal amplitude by an oscillating signal is in the range from 0.1 Hz to 5,040 Hz, wherein the impulse amplitude is in a range of from about 500 V to 30 kV, and wherein the electromagnetic field and high-frequency currents range from 40 MHz to 1 GHz and sound waves ranging from 0.1 to 20,000 Hz are generated through the generation of a dielectric barrier in an air gap between the transducer surface and the surface of the target treated tissue.

24. The device for treatment of diseases and conditions of claim 1, characterized in that the device generates impulse profiles that comprise impulse sequences that can be adapted and modulated based upon an impulse sequence frequency comprising a range from 0.1 to 7,500 Hz, the frequency of modulated impulses comprising a range from 20 to 350 kHz, and the frequency of the modulation signal comprising a range from 0.1 to 5,040 Hz.

25. The device for treatment of diseases and conditions of claim 24, characterized in that the impulse sequence frequency is one or more frequencies or a sweep over the frequency range range from 0.1 Hz to 7,500 Hz.

26. The device for treatment of diseases and conditions of claim 24, characterized in that the frequency of the modulated impulses is one or more frequencies or a sweep over the frequency range from 20 kHz to 350 kHz.

27. The device for treatment of diseases and conditions of claim 24, characterized in that the frequency of the modulation signal is one or more frequencies or a sweep over the frequency range from 0.1 to 5,040 Hz.

28. The device for treatment of diseases and conditions of claim 25, characterized in that the voltage on the secondary of the high-voltage transformer of the block comprising the generator and the signal profile regulator is generated as an impulse sequence whose waveform is either sinusoidal, rectangular, triangular, sawtooth, trapezoidal, linear or exponential growth or decay, even or odd harmonic, exponentially damped sine, or exponentially amplified sine, wherein the impulse sequences are modulated by sinusoidal, rectangular, trapezoidal, linear or exponential growth or decay, even or odd harmonics, or damped sinusoidal waveforms of the voltage managing signal wherein the voltage managing signal has a frequency ranging from 0.1 to 5,040 Hz and a modulation amplitude depth ranging from 1 to 100%.
